(19) United States
(12) Patent Application Publication
(10) Pub. No.: US 2018/0221161 A1
(43) Pub. Date: Aug. 9, 2018

(54) A JOINT PROSTHESIS

(71) Applicant: FIELD ORTHOPEDICS PTY LTD., St. Lucia (AU)

(72) Inventor: Chris Arnold Feffery, St. Lucia (AU)

(21) Appl. No.: 15/750,207

(22) PCT Filed: Aug. 13, 2016
(86) PCT No.: PCT/AU2016/000276

§ 371 Date: Feb. 5, 2018

(30) Foreign Application Priority Data
Aug. 13, 2015 (AU) 2015903255

(51) Int. Cl. A61F 2/42; A61F 2/30

(52) U.S. Cl. CPC A61F 2/4261; A61F 2/30749; A61F 2002/30126; A61F 2/4081; A61F 2002/30079; A61F 2/4225; A61F 2002/30125

(57) ABSTRACT

A joint prosthesis is provided that includes a proximal implant for attachment to a first portion of skeletal anatomy, the proximal implant including a proximal bone attachment portion and a distal cup portion. The prosthesis also includes a distal implant for attachment to a second portion of skeletal anatomy, the distal implant including a distal bone attachment portion and a proximal cup portion. An inter-joint element is located between the distal cup and the proximal cup. The inter-joint element includes surface portions shaped to complement said cups and is preferably formed as an ovoid. Each of the proximal implant, distal implant and inter-joint element include magnetic regions whereby the inter-joint element is attracted to both the proximal cup portion and to the distal cup portion to thereby provide stability across a large angular motion.

### TECHNICAL FIELD

The present invention relates to prosthetic joints and in a preferred embodiment to a wrist joint prosthesis.

### BACKGROUND

The discussion of any prior art documents, techniques, methods or apparatus is not to be taken to constitute any admission or evidence that such prior art forms, or ever formed, part of the common general knowledge.

Arthritis has a significant global burden documented to affect 1 in 7 Australians with prevalence increasing with the aging population. Common treatment modalities for severe arthritic disease in most joints of the body is via prosthetic replacement. In the hand however, despite being one of the most common collection of joints affected (up to 100 in 100,000) to date there has been minimal progress in the development of a physiologically equivalent implant.

FIG. 1 depicts the skeletal anatomy of the left wrist. Wrist replacement/arthroplasty is a surgical prosthesis implantation procedure to replace the diseased articulating surfaces of both the distal radius and one or more proximal carpal row surfaces in order to recreate the wrist joint and restore physiological function. Complexities in the realization of this prosthesis stem from the extreme range of motion of the articulating surfaces, minimal soft tissue supports and the high loads that are funneled through the surfaces during normal daily use.

Prior art prostheses to address this area of need have to date only been partially successful. Past technologies have focused on experimentation with varying surfaces materials and screw fixation and as such have been forced to design in mechanical constraints to ensure stability and subject the patient to ongoing lifestyle limitations to protect from failure. As such to date no past prosthesis has had the ability to recreate the stability through the full range of motion experience by the organic non-diseased joint and further to this, due to restricted nature of current prosthetics, true biologically equivalent wrist biomechanics has not yet been realized.

It is an object of the present invention to provide an improved joint prosthesis that is stable and which allows for natural movement over a wide range of angles.

### SUMMARY OF THE INVENTION

According to a first aspect of the present invention there is provided a joint prosthesis including:

a proximal implant for attachment to a first portion of skeletal anatomy, the proximal implant including a proximal bone attachment portion and a distal cup portion;

a distal implant for attachment to a second portion of skeletal anatomy, the distal implant including a distal bone attachment portion and a proximal cup portion;

an inter-joint element located between the distal cup and the proximal cup, the inter-joint element including surface portions shaped to complement said cups to allow radial movement of the proximal and distal cups relative to the inter-joint element wherein the inter-joint element is shaped to be retained in an internal space defined by the distal cup portion and the proximal cup portion; and the proximal and distal cup portions are configured to prevent contact between the inter-joint element and surrounding bone tissue during use;

wherein each of the proximal implant, distal implant and inter-joint element include magnetic regions whereby the inter-joint element is attracted to both the proximal cup portion and to the distal cup portion.

Preferably each of the distal cup and the proximal cup present concave articulation surfaces for articulating with the inter-joint element.

The inter-joint element is preferably ovoid or ellipsoid and the concave articulation surfaces of the distal cup and the proximal cup are correspondingly formed.

In a preferred embodiment of the invention the concave articulation surfaces are formed with a minor curvature and a major curvature for facilitating wrist flexion/extension and radial deviation respectively.

Preferably the proximal bone attachment portion comprises an elongate peg and the first portion of skeletal anatomy comprises a long bone, such as a radius, wherein the peg is shaped for insertion into a canal of the radius.

The distal bone attachment portion may comprise a peg for engagement into a hole formed into the capitate during implantation surgery.

The distal bone attachment portion may further include a mounting formation for attachment to a portion of the skeletal anatomy comprising a carpal bone.

According to a further aspect of the present invention there is provided a prosthesis for a human wrist comprising:

a proximal implant for attachment to a first portion of skeletal anatomy, the proximal implant including a proximal bone attachment portion and a distal cup portion, the distal cup portion presenting a concave articulation surface formed with different curvatures for wrist flexion/extension and for radial deviation;

a distal implant for attachment to a second portion of skeletal anatomy, the distal implant including a distal bone attachment portion and a proximal cup portion, the proximal cup portion presenting a concave articulation surface formed with said different curvatures for wrist flexion/extension and for radial deviation;

an inter-joint element located between the distal cup and the proximal cup shaped to articulate with said articulation surfaces of the cups wherein the inter-joint element is shaped to be retained in an internal space defined by the distal cup portion and the proximal cup portion thereby preventing contact between the inter-joint element and surrounding bone tissue during use; and;

wherein each of the proximal implant, distal implant and inter-joint element include magnetic regions whereby the inter-joint element is attracted to both the proximal cup portion and to the distal cup portion.

Preferably the inter-joint element has an ovoid or ellipsoid shape.

The distal bone attachment portion preferably comprises a peg for engagement into a hole formed into a distal carpal bone during implantation surgery.

In a preferred embodiment of the invention the distal bone attachment portion further includes a mounting formation for placement about a distal carpal bone and fastening thereto.

The peg is preferably arranged for engagement into a hole formed into a capitate bone during implantation surgery.

It is preferred that the distal bone attachment portion is arranged for engagement with a portion of trapezium and/or trapezoid bone during implantation surgery.

According to a further aspect of the present invention there is provided a method for wrist joint replacement of a patient by means of a wrist joint prosthesis as described herein, the method comprising the steps of:

excising the proximal carpal row bones of a hand of the patient;

inserting the peg of the proximal implant into a radius of the patient;

inserting the distal peg into a capitate bone of the hand.

Preferably the method further includes fastening the mounting formation of the distal bone attachment portion to a distal carpal bone of the patient.

### BRIEF DESCRIPTION OF THE DRAWINGS

The Detailed Description is not to be regarded as limiting the scope of the preceding Summary of the Invention in any way. The Detailed Description will make reference to a number of drawings as follows:

FIG. 1 is a diagram of the skeleton of the left wrist of an average adult male.

FIG. 2 is a somewhat exploded view of a prosthesis according to a preferred embodiment of the present invention.

FIG. 3 is a further, somewhat exploded view of the prosthesis of FIG. 2.

FIG. 4A is a schematic side view of the prosthesis of FIG. 2 at a first joint angle.

FIG. 4B is a schematic side view of the prosthesis of FIG. 2 at a second joint angle.

FIG. 5 is a somewhat stylized view showing the placement of the prosthesis relative to the bones of the hand of FIG. 1, post implantation.

FIG. 6 is a first side view of a foot skeleton showing the implantation of two joints according to embodiments of the present invention therein.

FIG. 7 is second side view of a foot skeleton showing the implantation of two joints according to embodiments of the present invention therein.

FIG. 8 is a view of the top of a skeleton of the foot showing the implantation of three joints according to embodiments of the present invention therein.

FIG. 9 is a view of a skeleton of a hand showing the implantation of seven joints according to embodiments of the present invention therein.

FIG. 10 is a view of a skeleton of a shoulder showing the implantation of a shoulder joint according to an embodiment of the present invention therein.

FIG. 11 is a first view of a human spinal skeleton showing the implantation of two joints according to embodiments of the present invention therein.

FIG. 12 is a side view of the implanted spinal skeleton of FIG. 11.

### DETAILED DESCRIPTION OF PREFERRED EMBODIMENTS

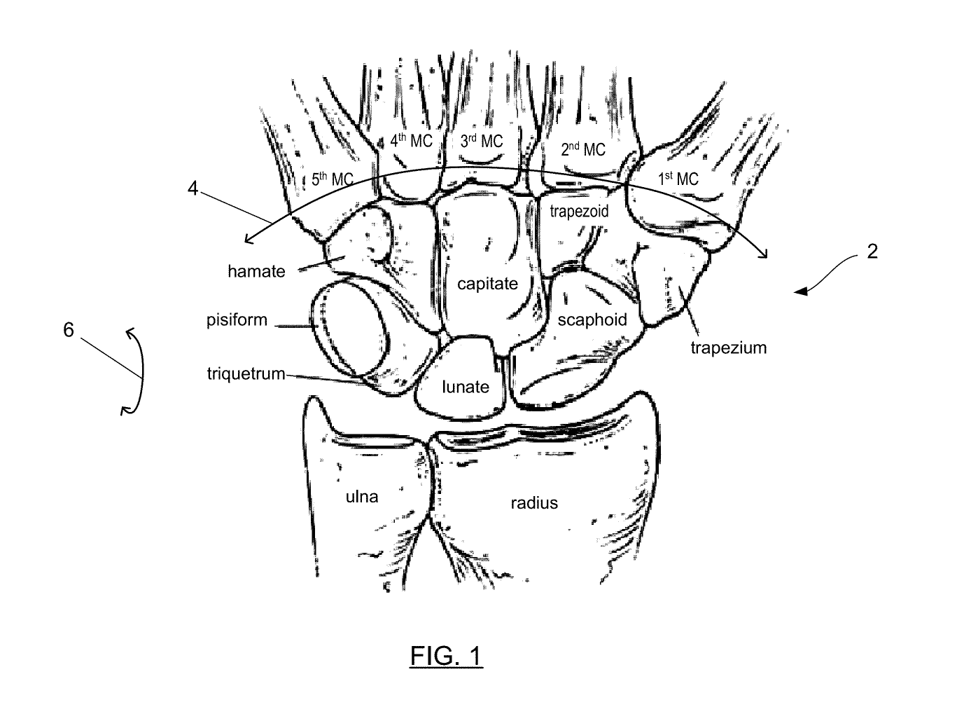

With reference to FIG. 1, it will be observed that the wrist joint 2 provides bi-articulation, namely radial deviation (or “panning”) of the hand in the direction indicated by arrow 4 and also wrist flexion and extension (or “tilting”) of the hand in the direction indicated by arrow 6. The Inventors have realized that to date a difficulty with creating physiologic motion and stability in wrist arthroplasty has been the limitation of attempting to reproduce the bi-articular articulation of the wrist with a mono articular implant. Mono articulation, i.e. having a single joint interface has been required in the past due to intra-component instability preventing the use of more than one joint interface.

The Inventors have conceived that the wrist joint can be viewed as a modified universal joint allowing flexion-extension and radial ulnar deviation. The flexion extension axis is typically 3-10 mm proximal to the radial-ulnar axis in an adult human.

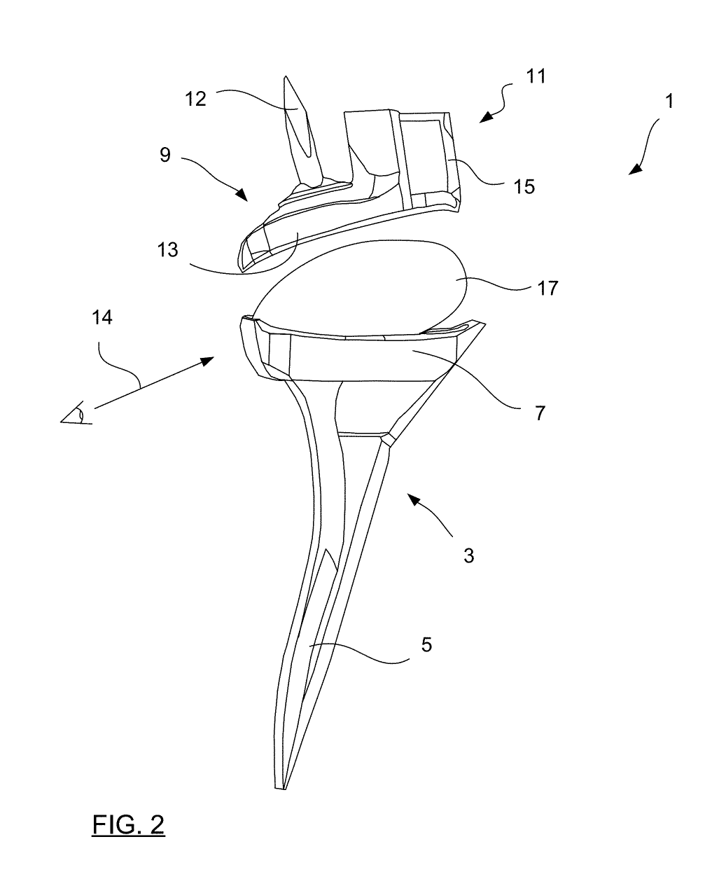

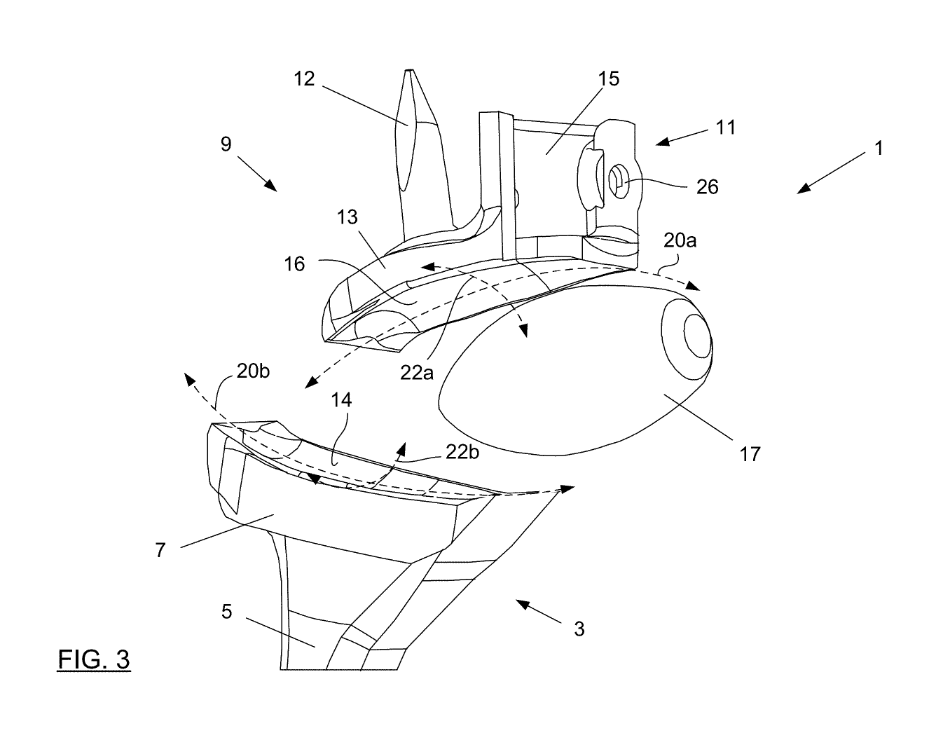

Referring now to FIG. 2, there is depicted a slightly exploded view of a joint prosthesis 1 according to a preferred embodiment of the present invention. FIG. 3 is a further, exploded view of the prosthesis of FIG. 2 from a second angle. The joint prosthesis 1 includes a proximal implant 3 for attachment to a first portion of skeletal anatomy such as the radius. The proximal implant 3 also includes a proximal bone attachment portion in the form of a first peg being a radial peg 5 for insertion into the radius bone of the arm and a distal cup portion in the form of cup 7.

Prosthesis 1 further includes a distal implant 9 for attachment to a second portion of skeletal anatomy, such as one or more of the distal carpal bones of the wrist. The distal implant 9 includes a distal bone attachment portion 11 and a proximal cup portion 13. The distal bone attachment portion 11 is comprised of a second peg in the form of a capitate peg 12 and a mounting formation 15 for attachment to one or more distal carpal bones of the wrist. For example, the mounting formation 15 may be attached to the trapezoid by locating the formation 15 about the proximal end of the trapezoid and then inserting a screw through hole 26 (visible in FIG. 3) and screwing it into the trapezoid.

The prosthesis 1 further includes an inter-joint element in the form of an ellipsoid or ovoid 17, which locates between the distal cup 13 and the proximal cup 7. The ovoid 17 is shaped to complement internal concave articulating surfaces 14 and 16 of the cups 7 and 13. The articulating surfaces 16, 14 each have a minor curvature as indicated by arrows 22a and 22b and a major curvature as indicated by arrows 20a and 20b. The minor curvature has a smaller radius of curvature than that of the major curvature. These curvatures 20a, 20b and 22a, 22b complement the shape of the ovoid 17 and respectively assist the implant in providing motions somewhat similar to the radial deviation motion 4 and the wrist flexion and extension motion 6 that are illustrated with reference to a normal wrist in FIG. 1. Whilst an ovoid or ellipsoid shaped inter-joint element is preferred for the wrist joint prosthesis 1, for some joints a generally spherical inter-joint element 17 and correspondingly shaped cups 7, 13, may be applicable.

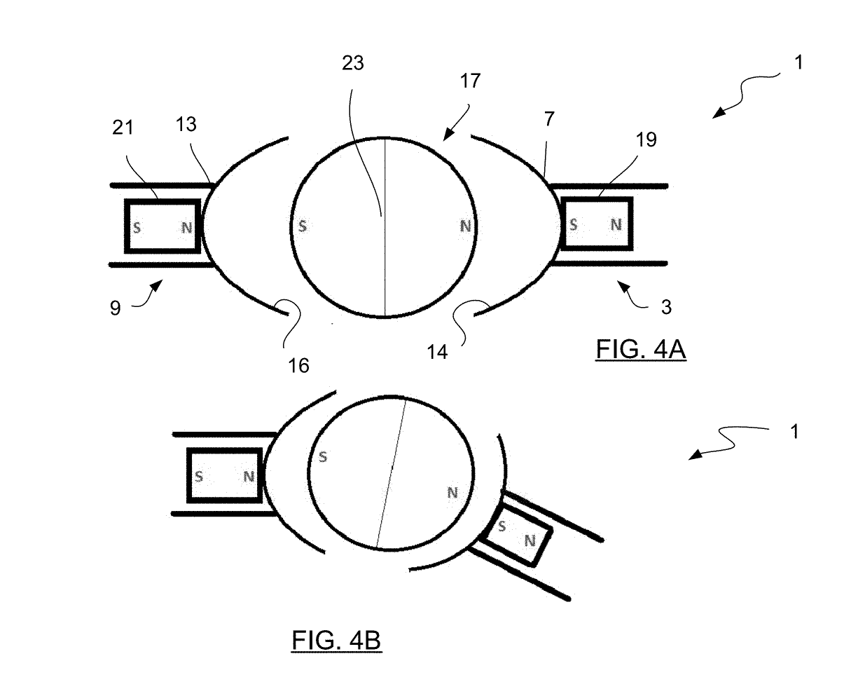

Referring now to FIGS. 4A and 4B, there is shown a schematic diagram of a mid-line cross section of the prosthesis 1 looking along the line of sight 14 that is indicated in FIG. 2. As shown in the schematic diagrams 4A and 4B of the prosthesis 1, each of the proximal implant 3, distal implant 9 and inter-joint element 17 include magnetic regions, respectively items 19, 21 and 23 so that the inter-joint element 17 is attracted to, and biased against, both the proximal cup portion 7 and the distal cup portion 13.

The magnets 19 21 and 23 preferably comprise neodymium magnets.

The inventors have found that by using magnets 19, 21 in both the implant portions 3, 9 of the prosthesis 1 as well as magnet 23 for the inter-joint element 17 a reliable magnetic coupling is provided. The magnetic coupling allows manipulation of the articulating surfaces 14, 16 of the cups 13, 7 with respect to the surface of the inter-joint element 17. The resultant manipulation can be made over a large range of motion whilst providing stability to avoid failure. For example, the prosthesis 1 remains stable in both the joint angle of FIG. 4A, which is approximately 180 degrees, and the joint angle of FIG. 4B, which is inclined at about 153 degrees.

The Inventors have conceived that the articulation of the articulating surfaces is a dynamic interaction in which there is a dramatic alteration in the area of opposition and as such there is a need for a dynamic axis of coupling to stabilize the wrist joint throughout the entire range of motion. This fundamental need is realized through the utilization of the three part magnetic system that is illustrated in FIGS. 4A and 4B. It will be observed that one magnet (19) is located adjacent the proximal articulating surface (i.e. cup 7), whilst another (magnet 21) is located adjacent the distal articulating surface (i.e. cup 13) and the third (magnet 23) is disposed within the joint space (i.e. within, or comprising of, the inter-cup element 17). As the joint moves throughout its range of motion the intra-articulating magnet, i.e. the ovoid 17, continually re-orientates itself to maintain magnetic alignment and thereby maintains the magnetic couple and joint stability.

Due to this persistence in the magnetic couple a prosthesis according to a preferred embodiment of the invention does not just ensure stability in the resting position, but allows the recreation of a joint that has enough stability at the extremes of range for true physiological motion to be realized.

The dual articulation, i.e. articulation in both the radial deviation direction corresponding to cup curvatures 20a and 20b and in the wrist flexion/extension direction corresponding to cup curvatures 22a, 22b, provides a prosthesis 1 that models normal wrist kinematics. The modelling of the normal wrist kinematics reduces stress across the joint 1 and allows a greater range of motion.

By aligning the axis of the distal articulation obliquely in the radial extension/ulnar flexion axis the prosthesis can be biased to allow motion in the so called “Dart Throwers” plane. This further reduces implant stresses by allowing motion in the most stable and physiologic plane.

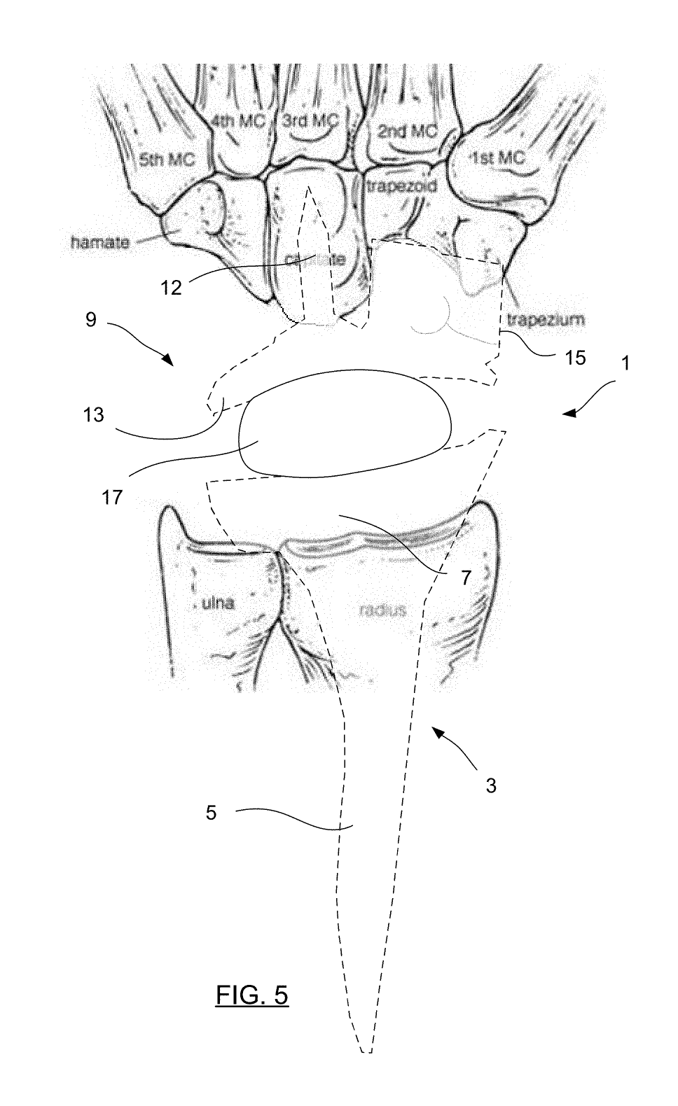

FIG. 5 is a somewhat stylized view showing the placement of the joint prosthesis 1 relative to the bones of the arm, i.e. the ulna and the radius and the wrist, post-implantation. A suitable surgical procedure to implant the prosthesis will now be explained.

Initially the wrist joint is exposed via a longitudinal 20 cm incision directed over Lister's tubercle on the dorsum of the radius. The subcutaneous veins are ligated. The extensor retinaculum is divided in a “z” fashion from the second compartment to the fifth compartment. The digital extensor tendons are retracted to the ulnar side and the radial wrist extensor tendons retracted to the radial side. The dorsal wrist capsule is elevated as a distal based rectangular flap from the articular margin of the radius and TFCC over the ulna extending along the ECU tendon on the ulnar side and over the radial border of the scaphoid on the radial side of the wrist.

The wrist is flexed and the proximal carpal row bones, i.e. the scaphoid, lunate, and triquetrum are excised preserving the volar extrinsic ligaments. The radius is instrumented with a guide pin to establish the longitudinal and central axis of the bone. The radius is prepared by drilling and broaching the canal and by milling the articular surface to match the radial component of the implant. The distal row is prepared in a similar fashion by establishing the longitudinal and central axis of the capitate and drilling and broaching the capitate to accept the peg of the base plate of the distal component of the implant. The proximal surfaces of the distal row bones are milled to prepare for the seating of the base plate. The articulations between the capitate and hamate, between the capitate and trapezoid and between the trapezoid and trapezium are prepared for formal arthrodesis by debriding the adjacent cartilage surfaces.

The radial implant (i.e. proximal implant 3) is then impacted. The distal implant 9 is placed and transverse drill holes are made through the distal row of bones. There is an option to screw distally into a carpal bone such as the trapezium and/or trapezoid.

The tension in the prosthesis is then checked with the central component, i.e. inter-joint element 17, placed. The tension can be adjusted by placing thicker or thinner components of the prosthesis 1 as required.

Definitive implantation is then undertaken and stability and range of motion checked.

The capsule is closed loosely with an absorbable suture. The extensor retinaculum is repaired with absorbable suture. The skin is closed over a surgical drain with non-absorbable sutures. The wrist is placed in a well-padded bandage and postoperative hand therapy commenced after two to three days.

Although a preferred embodiment of the invention has been described for use with the wrist, the invention is directly relevant for application to many joints of the body, as well as retrofitting into existing prosthesis to increase stability.

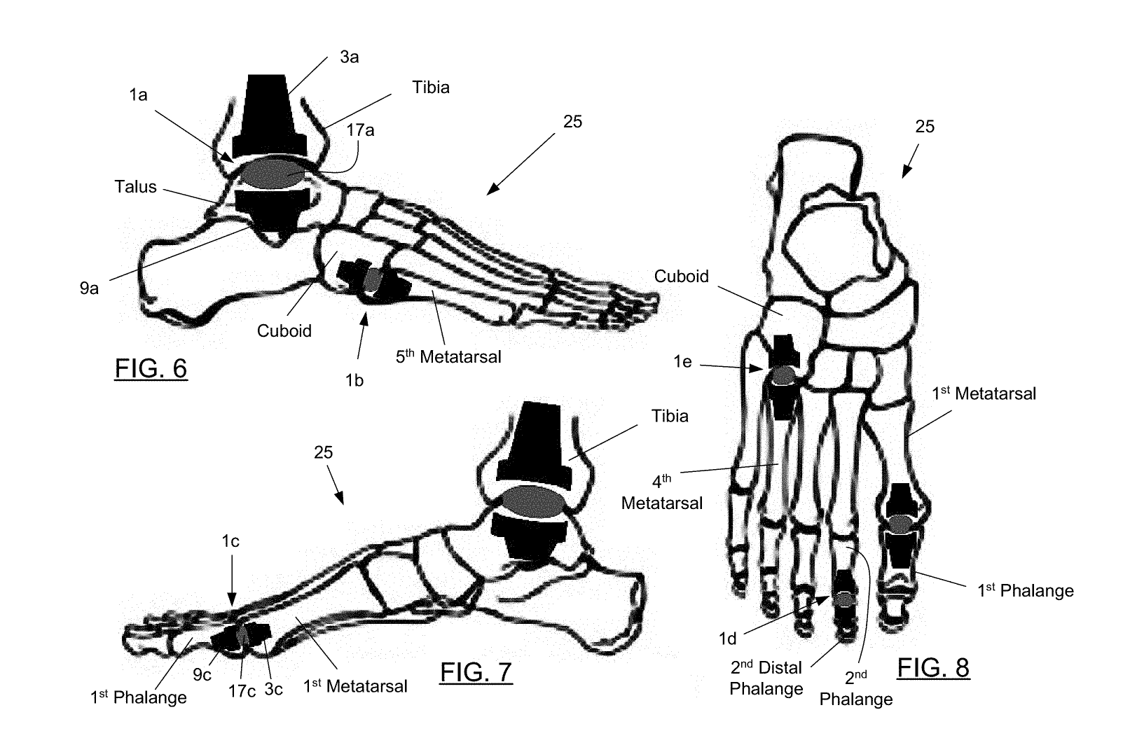

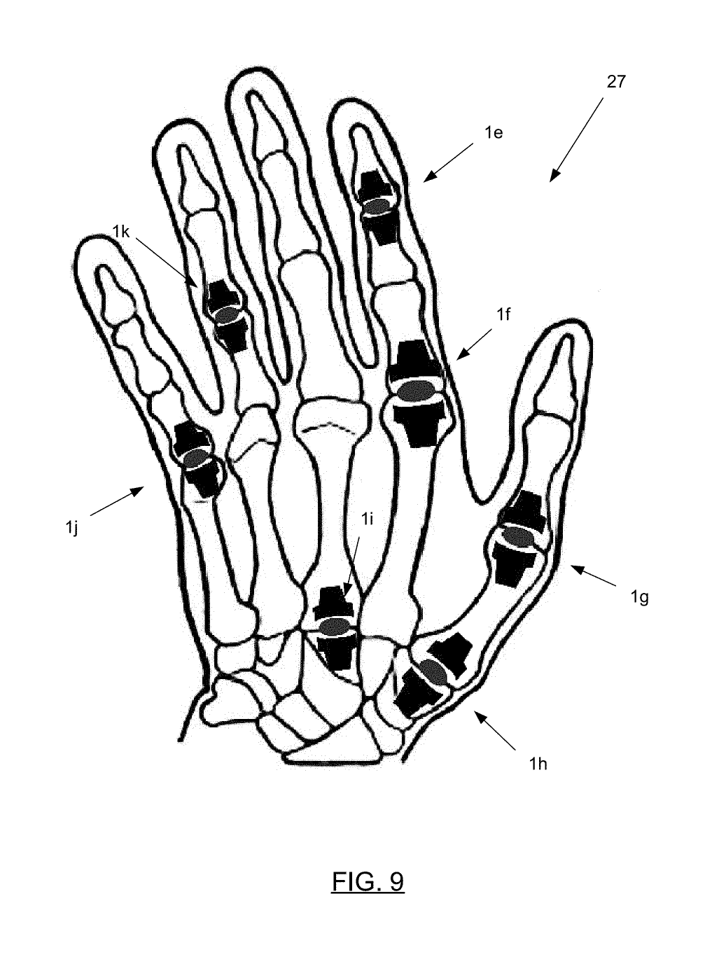

For example, FIGS. 6, 7 and 8 depict various joints according to embodiments of the present invention implanted into a human adult foot 25. With reference to FIG. 6 a joint 1a is interconnected between the tibia bone of the lower leg and the talus bone of the foot 25. A smaller joint 1b is interconnected between the cuboid bone and the 5th metatarsal. FIG. 7 shows the placement of a further joint 1c interconnecting the 1^{st} metatarsal and the 1^{st} phalange bones. FIG. 8 shows the connection of joint 1d between the 2^{nd} distal phalange and the 2^{nd} phalange and also the connection of joint 1e between the cuboid bone and the 4^{th} metatarsal. In each situation the first and second pegs of each joint are appropriately dimensioned and sized to be received by their respective target bones, e.g. the tibia and talus in the case of joint 1a.

FIG. 9 shows the skeleton of a hand 27 in which a number of joint implants 1e to 1k have been located between various bones of the hand to replace arthritic joints. In each case the joints are sized to be appropriate to the anatomy of the bones involved.

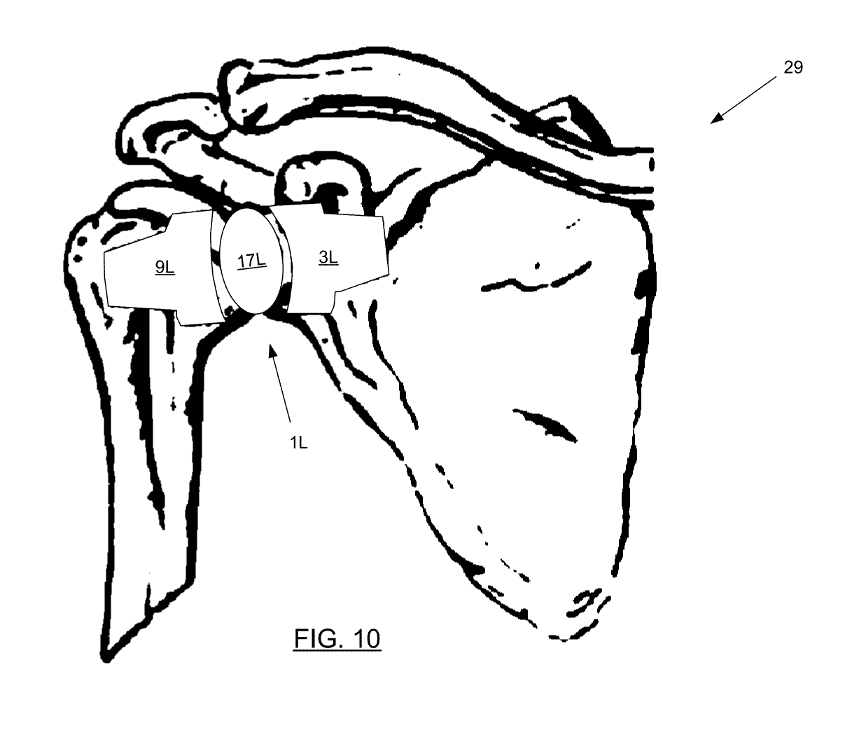

FIG. 10 shows an implant 1L according to an embodiment of the present invention implanted to implement a shoulder joint in a human shoulder 29.

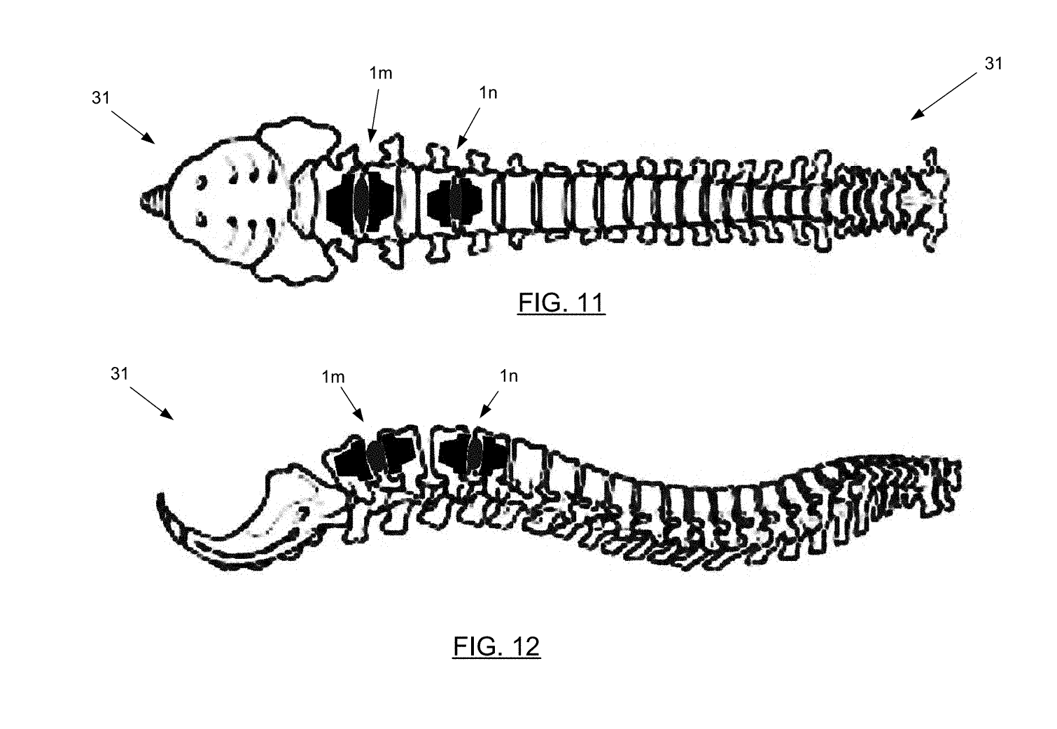

FIGS. 11 and 12 are plan and side views of a human spine skeleton showing how joints 1m and 1n according to embodiments of the present invention have been used to implement inter-vertebral joints in the lumber region pf the spine.

Additionally whilst the preferred embodiment of the invention has been described in the context of human use it is also relevant for animal joints and for creation of artificial joints for robotic and bionic applications—wherever there is a requirement to artificially construct a stable joint.

In compliance with the statute, the invention has been described in language more or less specific to structural or methodical features. The term “comprises” and its variations, such as “comprising” and “comprised of” is used throughout in an inclusive sense and not to the exclusion of any additional features.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A joint prosthesis including:
a proximal implant for attachment to a first portion of skeletal anatomy, the proximal implant including a proximal bone attachment portion and a distal cup portion;
a distal implant for attachment to a second portion of skeletal anatomy, the distal implant including a distal bone attachment portion and a proximal cup portion;
an inter-joint element located between the distal cup and the proximal cup, the inter-joint element including surface portions shaped to complement said cups to allow radial movement of the proximal and distal cups relative to the inter-joint element wherein the inter-joint element is shaped to be retained in an internal space defined by the distal cup portion and the proximal cup portion; and the proximal and distal cup portions are configured to prevent contact between the inter-joint element and surrounding bone tissue during use;
wherein each of the proximal implant, distal implant and inter-joint element include magnetic regions whereby the inter-joint element is attracted to both the proximal cup portion and to the distal cup portion.

2. A joint prosthesis according to claim 1, wherein each of the distal cup and the proximal cup present concave articulation surfaces for articulating with the inter-joint element.

3. A joint prosthesis according to claim 2, wherein the inter-joint element is ovoid or ellipsoid and the concave articulation surfaces of the distal cup and the proximal cup are correspondingly formed.

4. A joint prosthesis according to claim 2, wherein the concave articulation surfaces are formed with a minor curvature and a major curvature for facilitating wrist flexion/extension and radial deviation respectively.

5. A joint prosthesis according to claim 1 wherein the proximal bone attachment portion comprises an elongate peg and the first portion of skeletal anatomy comprises a long bone, such as a radius, wherein the peg is shaped for insertion into a canal of the radius.

6. A joint prosthesis according to claim 1 wherein the distal bone attachment portion comprises a peg for engagement into a hole formed into the capitate during implantation surgery.

7. A joint prosthesis according to claim 6, wherein the distal bone attachment portion further includes a mounting formation for attachment to a portion of the skeletal anatomy comprising a carpal bone.

8. A prosthesis for a human wrist comprising:
a proximal implant for attachment to a first portion of skeletal anatomy, the proximal implant including a proximal bone attachment portion and a distal cup portion, the distal cup portion presenting a concave articulation surface formed with different curvatures for wrist flexion/extension and for radial deviation;
a distal implant for attachment to a second portion of skeletal anatomy, the distal implant including a distal bone attachment portion and a proximal cup portion, the proximal cup portion presenting a concave articulation surface formed with said different curvatures for wrist flexion/extension and for radial deviation;
an inter-joint element located between the distal cup and the proximal cup shaped to articulate with said articulation surfaces of the cups wherein the inter-joint element is shaped to be retained in an internal space defined by the distal cup portion and the proximal cup portion thereby preventing contact between the inter-joint element and surrounding bone tissue during use; and
wherein each of the proximal implant, distal implant and inter-joint element include magnetic regions whereby the inter-joint element is attracted to both the proximal cup portion and to the distal cup portion.

9. A prosthesis according to claim 8 wherein the inter-joint element has an ovoid or ellipsoid shape.

10. A prosthesis according to claim 8 wherein the distal bone attachment portion comprises a peg for engagement into a hole formed into a distal carpal bone during implantation surgery.

11. A prosthesis for a human wrist according to claim 8 wherein the distal bone attachment portion further includes a mounting formation for placement about a distal carpal bone and fastening thereto.

12. A prosthesis according to claim 10 wherein the peg is arranged for engagement into a hole formed into a capitate bone during implantation surgery.

13. A prosthesis according to claim 11, wherein the distal bone attachment portion is arranged for engagement with a portion of trapezium and/or trapezoid bone during implantation surgery.

14. A method for wrist joint replacement of a patient by means of a wrist joint prosthesis according to claim 8, the method comprising the steps of:
excising the proximal carpal row bones of a hand of the patient;
inserting the peg of the proximal implant into a radius of the patient;
inserting the distal peg into a capitate bone of the hand.

15. The method of claim 14, further including fastening the mounting formation of the distal bone attachment portion to a distal carpal bone of the patient.
